(19) United States
(12) Patent Application Publication
(10) Pub. No.: US 2025/0135240 A1
(43) Pub. Date: May 1, 2025

(54) HIGH-INTENSITY FOCUSED ULTRASOUND GENERATOR USING INSULATING MATERIAL

(71) Applicant: JEISYS MEDICAL INC., Seoul (KR)

(72) Inventors: Won Ju Yi, Seoul (KR); Eun Kyung Gong, Seoul (KR); Soo Min Hwang, Seoul (KR); Dong Hwan Kang, Seoul (KR)

(73) Assignee: JEISYS MEDICAL INC., Seoul (KR)

(21) Appl. No.: 19/004,487

(22) Filed: Dec. 30, 2024

Related U.S. Application Data
(63) Continuation of application No. PCT/KR2023/008040, filed on Jun. 12, 2023.

(30) Foreign Application Priority Data
Jul. 5, 2022 (KR) 10-2022-0082473
Jul. 5, 2022 (KR) 10-2022-0082474

(51) Int. Cl. A61N 7/02; B06B 1/06

(52) U.S. Cl. CPC A61N 7/02; B06B 1/0625

(57) ABSTRACT

A plurality of transducers are individually mounted to an ultrasonic radiation frame by transducer holders, and at least a portion of the transducer holders is composed of electrodes made of a conductive material. The front surfaces of the transducers contact the transducer holders and are electrically connected thereto, and the rear surfaces of the transducers are electrically connected to the transducer holders through electrode wires such that there is no need to solder the electrode wires to the front surfaces of the transducers, and thus water leakage due to a soldered structure can be prevented, and manufacturing can be more convenient. In addition, the transducers are configured to sit on support projections of the transducer holders such that the electrode wires coupled to the lower surfaces of the transducers are prevented from contacting the transducer holders.

### CROSS-REFERENCE TO RELATED APPLICATIONS

The present application is a continuation of International Patent Application No. PCT/KR2023/008040, filed on Jun. 12, 2023, which claims the benefit of priority to Korean Patent Application Nos. 10-2022-0082473 filed on Jul. 5, 2022 and 10-2022-0082474 filed on Jul. 5, 2022. The disclosures of the above-listed applications are hereby incorporated by reference herein in their entirety.

### FIELD

The present disclosure relates to a high-intensity focused ultrasound generator using an insulating material in which a plurality of transducers are individually mounted on an ultrasonic radiation frame by a transducer holder, and at least a part of the ultrasonic radiation frame is made of an insulating material, thereby improving durability and insulation.

### BACKGROUND

In general, a high-intensity focused ultrasound (HIFU) generator is a device that focuses ultrasound generated from a transducer to generate high-intensity ultrasound energy, irradiates it to the patient's affected area, and increases the temperature of the affected area, thereby treating the affected area without surgical intervention.

In a conventional high-intensity focused ultrasound generator, when tens or hundreds of transducers are used, a plurality of transducers is mounted on a front of an ultrasound radiation frame, and then the entire front of the ultrasound radiation frame is coated with glue to form a waterproof layer, thereby fixing the plurality of transducers by the waterproof layer and preventing water leakage.

However, since the ultrasound energy generated forward from the transducers is absorbed by the waterproof layer, there is a problem that the input voltage needs to be increased to compensate for this, and there is also a problem that the ultrasound radiation frame needs be replaced even in the case that any one of the plurality of transducers breaks down.

### SUMMARY

In an exemplary embodiment, the present disclosure provides a high-intensity focused ultrasound generator. The high-intensity focused ultrasound generator includes: an ultrasonic radiation frame and a coupling hole formed therein; a transducer holder inserted into the coupling hole of the ultrasonic radiation frame and detachably coupled through the ultrasonic radiation frame; and a transducer mounted on the transducer holder. The ultrasonic radiation frame comprises an insulating material. The transducer holder is an electrode formed of a conductive material or an electrode comprising a surface coated with a conductive material. At least one of a front surface or a side surface of the transducer is electrically coupled to the transducer holder. A rear surface of the transducer has an electrical coupling through a current supply hole formed in a rear surface of the transducer holder.

### BRIEF DESCRIPTION OF THE FIGURES

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a perspective view illustrating a head module of a high-intensity focused ultrasound generator according to a first embodiment of the present disclosure.

FIG. 2 is an exploded perspective view illustrating a combined structure of an ultrasonic radiation frame and a transducer holder according to the first embodiment of the present disclosure.

FIG. 3 is a cross-sectional view illustrating a coupling structure of an ultrasonic radiation frame and a transducer holder according to the first embodiment of the present disclosure.

FIG. 4 is an enlarged view of part A of FIG. 3.

FIG. 5 a front perspective view of a transducer holder according to the first embodiment of the present disclosure.

FIG. 6 a rear perspective view of the transducer holder shown in FIG. 5.

FIG. 7 is a diagram illustrating an electrode structure using a transducer holder according to a second embodiment of the present disclosure.

FIG. 8 is a rear view illustrating an ultrasonic radiation frame and an RF board according to a fourth embodiment of the present disclosure.

FIG. 9 is a cross-sectional view taken along line A-A of FIG. 8.

### DETAILED DESCRIPTION

Exemplary embodiments of the present disclosure provide a high-intensity focused ultrasound generator using an insulating material that is easy to be replaced and repaired and ensures durability and insulation of the transducer.

A high-intensity focused ultrasound generator using an insulating material according to an aspect of the present disclosure includes an ultrasonic radiation frame having a concave front surface and a plurality of coupling holes formed therein; a plurality of transducer holders respectively inserted into the plurality of coupling holes in a front of the ultrasonic radiation frame and detachably coupled through the ultrasonic radiation frame; and a plurality of transducers respectively mounted on the plurality of transducer holders with front surfaces thereof exposed, wherein the ultrasonic radiation frame is made of an insulating material, wherein the transducer holder is an electrode formed of a conductive material or an electrode formed by coating a surface with a conductive material, wherein at least one of a front surface or a side surface of the transducer is electrically connected by contacting the electrode, and wherein a rear surface of the transducer is coupled to a current supply unit inserted through a current supply hole formed in a rear surface of the transducer holder, so that current is supplied to the transducer by a potential difference applied to the electrode and the current supply unit.

The ultrasonic radiation frame is formed of resin or a carbon composite material including a carbon material mixed with the resin at a preset ratio.

The ultrasonic radiation frame includes a frame body portion formed of a metal, and an insulating layer formed by anodizing a surface of the frame body portion.

The surface of the ultrasonic radiation frame includes an insulating layer coated with an insulating material.

The transducer holder includes a head portion mounted on the front surface of the ultrasonic radiation frame and has a mounting groove formed into which the transducer is inserted and mounted, and a body portion that extends rearward from the head portion and penetrates the coupling hole and is coupled by a fastening member at a rear of the ultrasonic radiation frame.

The body portion of the transducer holder is formed with the current supply hole so that the current supply portion passes through and is withdrawn to the rear of the ultrasonic radiation frame, and a space between the current supply portion and the current supply hole is sealed with waterproof glue.

The head portion of the transducer holder is formed so that at least a part of a side surface of the mounting groove is opened.

The head portion of the transducer holder is formed with at least one support protrusion that protrudes from a bottom surface of the mounting groove and supports a lower surface of the transducer and form a space between the transducer and the bottom surface.

The head portion of the transducer holder is formed with a catch protrusion that protrudes from a bottom surface of the mounting groove and has a tip bent inward to prevent the transducer inserted into the mounting groove from being detached.

The body portion of the transducer holder includes a shaft portion that extends rearward from the head portion and is press-fitted into the coupling hole, and a screw portion that extends rearward from the shaft portion and penetrates the coupling hole and is coupled with the fastening member at the rear of the ultrasonic radiation frame.

The support protrusion is formed of a non-conductive material.

The side surface and the rear surface of the transducer are coated with at least one of a waterproof material or a non-conductive material.

A high-intensity focused ultrasound generator using an insulating material according to another aspect of the present disclosure includes an ultrasonic radiation frame having a probe arranged in a front center and a plurality of coupling holes formed around the probe; a plurality of transducer holders respectively inserted into the plurality of coupling holes in front of the ultrasonic radiation frame, penetrating the ultrasonic radiation frame, and detachably coupled at a rear of the ultrasonic radiation frame; and a plurality of transducers respectively mounted on open front surfaces of at least some of the plurality of transducer holders, wherein the ultrasonic radiation frame is made of an insulating material, wherein the transducer holder includes: a head portion mounted on a front surface of the ultrasonic radiation frame and has a mounting groove formed into which the transducer is inserted and mounted; and a body portion that extends rearward from the head portion and penetrates the coupling hole and is coupled by a fastening member at the rear of the ultrasonic radiation frame, wherein the transducer holder includes: a head portion that is formed with a mounting groove that is mounted on the front surface of the ultrasonic radiation frame and into which the transducer is inserted and mounted, and a body portion that extends rearward from the head portion and penetrates the coupling hole and is formed to be coupled by a fastening member at a rear of the ultrasonic radiation frame, wherein the head portion has a plurality of support protrusions that protrudes from a bottom surface of the mounting groove and supports a lower surface of the transducer and form a space between the transducer and the bottom surface, wherein the transducer holder is an electrode formed of a conductive material or an electrode formed by coating the surface with a conductive material, wherein at least one of a front surface and a side surface of the transducer is in contact with the electrode, wherein the rear surface of the transducer is coupled to an electrode wire inserted through an electrode wire hole formed in the body portion, so that current is supplied to the transducer by a potential difference applied to the electrode and the current supply unit, and further including an RF board provided on the ultrasonic radiation frame, and in which the plurality of transducers are electrically connected to each other and supply RF power to the transducers.

The current supply unit includes a plurality of electrode wires respectively connected to the plurality of transducers, and the RF board includes a plurality of board connectors respectively provided corresponding to the electrode wires, and to which the electrode wires are detachably connected.

The plurality of board connectors are detachably connected to the RF board.

The high-intensity focused ultrasound generator further includes a monitoring sensor provided on the RF board and configured to independently monitor operating states of the transducers by detecting the power supply states of the electrode wires respectively connected to the transducers.

The high-intensity focused ultrasound generator further includes an insulating cover formed to cover an outer side of the RF board.

The high-intensity focused ultrasound generator further includes a power supply device for supplying the RF power to the RF board, and a power cable for connecting the RF board and the power supply device and detachably coupled with the RF board.

The high-intensity focused ultrasound generator further includes a probe coupled to a center of the ultrasonic radiation frame.

The RF board is provided on a remaining part of the rear of the ultrasonic radiation frame except for a coupling portion where the probe is coupled.

At least one of the rear surface or the side surface of the transducer and the transducer holder are bonded by an adhesive member so that the transducer is sealed while being vibrated inside the transducer holder, and the transducer holder and the ultrasonic radiation frame are sealed by a sealing member.

A high-intensity focused ultrasound generator using an insulating material according to still another aspect of the present disclosure includes an ultrasonic radiation frame having a probe arranged in a front center and a plurality of coupling holes formed around the probe; a plurality of transducer holders respectively inserted into the plurality of coupling holes in front of the ultrasonic radiation frame, penetrating the ultrasonic radiation frame, and detachably coupled at a rear of the ultrasonic radiation frame; and a plurality of transducers respectively mounted on open front surfaces of at least some of the plurality of transducer holders, wherein the ultrasonic radiation frame is made of an insulating material, wherein the transducer holder is an electrode formed of a conductive material or an electrode formed by coating the surface with a conductive material, further comprising: an RF board provided on the ultrasonic radiation frame and supply RF power to the transducers; a plurality of electrode wires respectively connected to the plurality of transducers; a plurality of board connectors respectively provided corresponding to the electrode wires, and to which the electrode wires are detachably connected; and a monitoring sensor provided on the RF board and configured to independently monitor operating states of the transducers by detecting the power supply states of the electrode wires respectively connected to the transducers.

A high-intensity focused ultrasound generator using an insulating material according to still another aspect of the present disclosure includes an ultrasonic radiation frame having a concave front surface and a plurality of coupling holes formed therein; a plurality of transducer holders respectively inserted into the plurality of coupling holes in a front of the ultrasonic radiation frame and detachably coupled through the ultrasonic radiation frame; and a plurality of transducers respectively mounted on the plurality of transducer holders with front surfaces thereof exposed, wherein the ultrasonic radiation frame is made of an insulating material,

The transducer holder is an electrode formed of a conductive material or an electrode formed by coating a surface with a conductive material, further comprising: an RF board provided on the ultrasonic radiation frame, and in which the plurality of transducers are electrically connected to each other and supply RF power to the transducers; a plurality of electrode wires respectively connected to the plurality of transducers; a plurality of board connectors respectively provided corresponding to the electrode wires, and to which the electrode wires are detachably connected; a monitoring sensor provided on the RF board and configured to independently monitor operating states of the transducers by detecting the power supply states of the electrode wires respectively connected to the transducers; and an insulating cover formed to cover an outer side of the RF board, wherein the transducer holder includes: a head portion that is formed with a mounting groove that is mounted on the front surface of the ultrasonic radiation frame and into which the transducer is inserted and mounted, and a body portion that extends rearward from the head portion and penetrates the coupling hole and is formed to be coupled by a fastening member at a rear of the ultrasonic radiation frame, and wherein the body portion of the transducer holder is formed with the current supply hole so that the current supply portion passes through and is withdrawn to the rear of the ultrasonic radiation frame.

Hereinafter, exemplary embodiments of the present disclosure will be described with reference to the attached drawings.

A high-intensity focused ultrasound generator using an insulating material according to an embodiment of the present disclosure is a device using high-intensity focused ultrasound (HIFU). The high-intensity focused ultrasound generator includes a transducer array in which tens or hundreds of transducers are radially arranged, and may not only treat the affected area of a patient with a tumor, but also stimulate the brain to treat Alzheimer's disease or depression, and may also increase immunity by applying heat to a specific area.

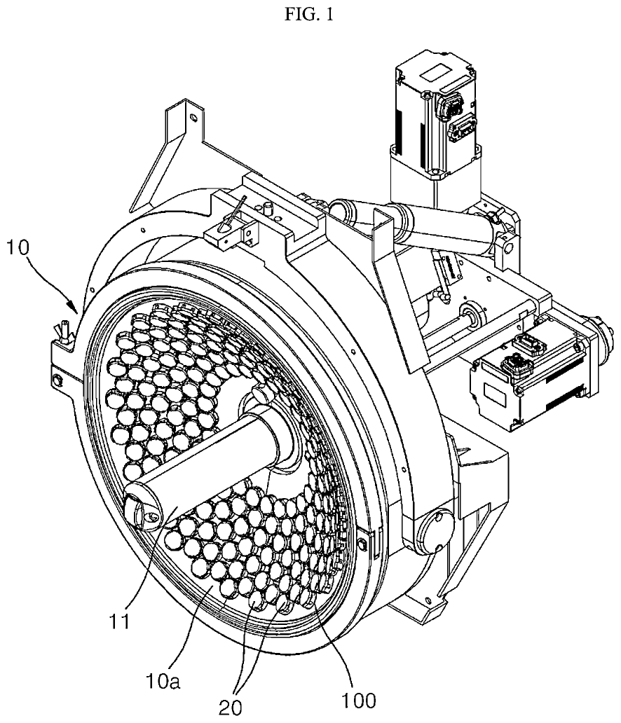

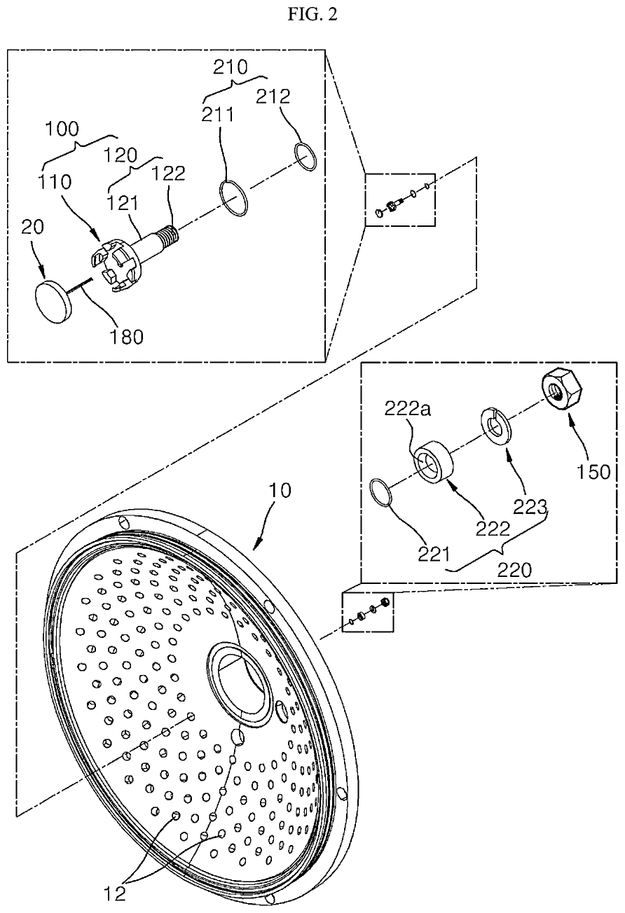

FIG. 1 is a perspective view illustrating a head module of a high-intensity focused ultrasound generator according to a first embodiment of the present disclosure. FIG. 2 is an exploded perspective view illustrating a combined structure of an ultrasonic radiation frame and a transducer holder according to the first embodiment of the present disclosure.

Referring to FIGS. 1 and 2, a head module of a high-intensity focused ultrasound generator includes an ultrasonic radiation frame 10, a plurality of transducers 20, and a plurality of transducer holders 100.

The ultrasonic radiation frame 10 is made of an insulating material. That is, the ultrasonic radiation frame 10 may be formed only of a resin, or may be formed of a carbon composite material in which a carbon material is mixed with the resin at a preset ratio. Here, the resin may include at least one of polyamide resin, acetal resin, polycarbonate, or polyphenylene oxide. The carbon material may include carbon fiber, carbon nanotube, graphite, graphene, and the like. The above-mentioned set ratio is a ratio set to limit the addition of the carbon material so that the carbon composite does not have a conductive property. The above-mentioned set ratio may be set in advance through experiments, and the like.

The ultrasonic radiation frame 10 has a probe 11 attached to a center of a front 10a, and a plurality of coupling holes 12 are arranged radially around the probe 11. The ultrasonic radiation frame 10 is formed in a plate shape with a concave front so as to focus ultrasound radiated from the plurality of transducers 20 and radiate them to one position.

The plurality of coupling holes 12 are through holes formed at a predetermined interval from each other. The number of the coupling holes 12 is set according to the number of the transducers 20.

The plurality of transducers 20 may include piezoelectric elements. The transducer 20 generates ultrasound when voltage is applied. The transducer 20 is formed in a disk shape, for example. The transducers 20 are arranged in a radial pattern in tens or hundreds to form a transducer array. The number of the transducers 20 may be set according to the ultrasonic energy to be radiated.

The transducers 20 are coated with an electrode material that is a conductive material on each surface. That is, the inside of each of the transducers 20 is made of a piezoelectric element material, and the surface is coated with an electrode material.

The transducer holder 100 is formed to have electrical conductivity, for example. In this embodiment, the transducer holder 100 is an example in which each surface is coated with a conductive material after being formed with a non-conductive material. The transducer holder 100 is an example in which the transducer holder is formed with a resin material and the surface is coated with the conductive material. In addition, only the surface of the transducer holder 100 excluding the inner side of the head portion 110 described below or the support protrusion 110b described below may be coated with the conductive material. In the case that the entire surface of the transducer holder 100 is coated with the conductive material, at least a portion of a side surface or a rear surface of the transducer 20 is coated with the non-conductive material to prevent short circuiting. In addition, in the case that at least a portion of the side surface and the rear surface of the transducer 20 is coated with a waterproof material, corrosion due to water intrusion and fluctuations in output values may be prevented.

The conductive material includes at least one of chromium, nickel, cadmium, iron, copper, platinum, gold, silver, lead, or an alloy, for example. However, the present disclosure is not limited thereto, and any material having electrical conductivity may be applied.

In this embodiment, a chromium coating layer is formed on the surface of the transducer holder 100 to form an electrode 170, for example.

The transducer holder 100 is detachably coupled to each of the plurality of coupling holes 12. The transducer 20 is coupled to each of the transducer holders 100.

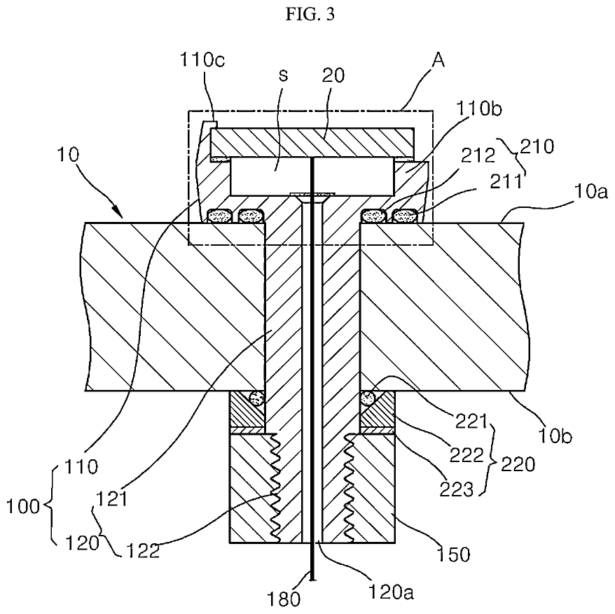

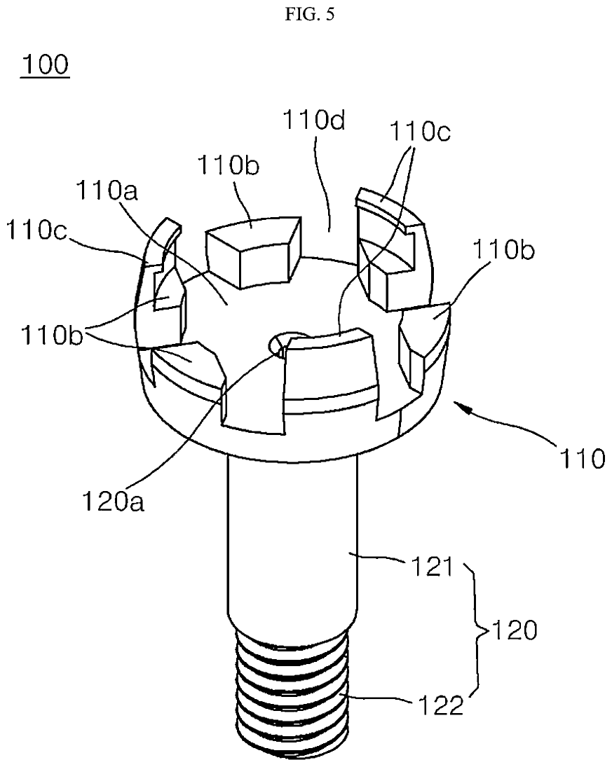

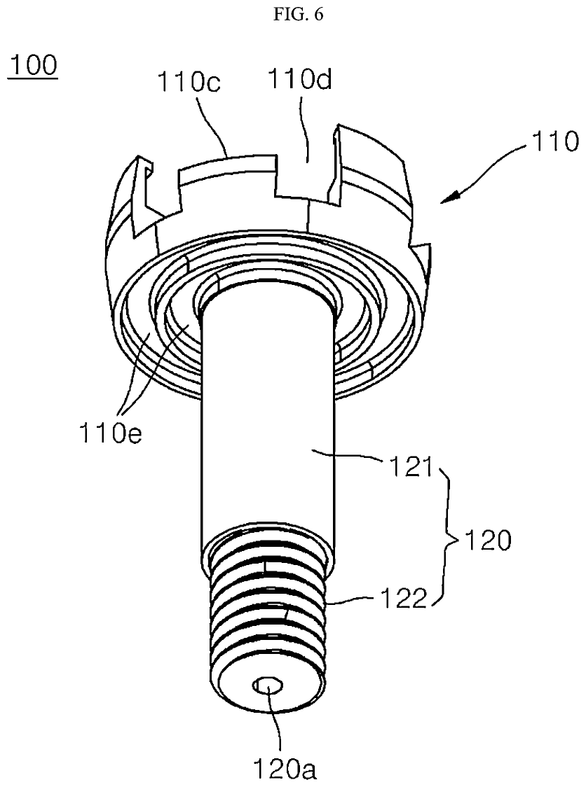

Referring to FIGS. 3 to 6, the transducer holder 100 includes a head portion 110 having a mounting groove 110a formed in which the transducer 20 is inserted and mounted, and a body portion 120 that extends to a rear of the head portion 110 and is coupled to the coupling holes 12.

The head portion 110 is formed to have a diameter greater than the coupling hole 12 so that the head portion 110 is mounted on the front 10a of the ultrasonic radiation frame 10. The mounting groove 110a, support protrusion 110b, a catch 110c, and an opening 110d are formed in the head portion 110.

The mounting groove 110a is formed with an open front in front of the head portion 110 so that the transducer 20 is mounted therein.

The support protrusion 110b is formed to protrude forward from the bottom surface of the mounting groove 110a to a predetermined height so as to support the lower surface of the transducer 20. The support protrusion 110b forms a space S between the lower surface of the transducer 20 and the bottom surface of the mounting groove 110a, thereby forming a passage through which the electrode wire 180 coupled to the transducer 20 passes, thereby stably implementing the electrode structure, and also enables vibration of the transducer 20 so as to maximize the vibration wave energy of the transducer 20. The above support protrusion 110b is composed of multiple pieces and is formed to be spaced apart from each other by a predetermined interval, as an example. However, the present disclosure is not limited thereto, and the support protrusion 110b may also be provided with one piece in the center of the bottom surface of the mounting groove 110a. In addition, the support protrusion 110b may also be formed integrally with the head portion 110, and of course, support protrusion 110b may also be formed by applying a non-conductive material, for example, glue, to the bottom surface of the mounting groove 110a.

The catch protrusion 110c is formed to protrude from the bottom surface of the mounting groove 110a and have a tip bent inward, thereby preventing the transducer 20 inserted into the mounting groove 110a from being detached. The tip of the catch protrusion 110c may be changed to a shape that may prevent the transducer 20 from being detached, such as a hook shape. The catch protrusion 110c is formed in a plurality of pieces spaced apart from each other by a predetermined interval. In this embodiment, some of the plurality of catch protrusions 110c may be formed by protruding from the support protrusion 110b.

The opening 110d is a part formed by being cut open in the side of the mounting groove 110a. The opening 110d has the advantage of easy assembly. In addition, the opening 110d allows the transducer 20 to vibrate inside the mounting groove 110a, thereby maximizing the vibration wave energy of the transducer 20.

It is preferable that the body portion 120 is formed to extend rearwardly from the head portion 110 and penetrates the coupling hole 12. The body portion 120 is formed to have a diameter smaller than that of the head portion 110. A current supply hole is formed in the center of the body portion 120 so that a current supply portion, described below, may pass through the current supply hole.

The body portion 120 includes a shaft portion 121 and a screw portion 122.

The shaft portion 121 is formed in a cylindrical shape so as to extend rearwardly from the head portion 110 and be pressed into the coupling hole 12.

The screw portion 122 is formed with threads on the outer surface so as to extend rearwardly from the shaft portion 121 and be fastened by a fastening member 150.

The fastening member 150 is preferably a nut, but is not limited thereto.

Meanwhile, FIG. 3 is a cross-sectional view illustrating a coupling structure of an ultrasonic radiation frame and a transducer holder according to the first embodiment of the present disclosure.

The adhesive material may be a flexible glue. A flexible glue layer 200 is formed between at least one of the rear surface or the side surface of the transducer 20 and the head portion 110 by the flexible glue. The flexible glue may use a silicone or epoxy-based glue, and any flexible material may be used.

In this embodiment, the flexible glue layer 200 may be formed between the rear surface of the transducer 20 and the support protrusion 110b. However, the present disclosure is not limited thereto, and the flexible glue layer 200 may also be formed between the side surface of the transducer 20 and the inner surface of the catch protrusion 110c. That is, the flexible glue layer 200 may be applied to any position so long as the flexible glue layer 200 does not cover the front of the transducer 20.

Since the transducer 20 is fixed by being adhered to the transducer holder 100 by the flexible glue, the position of the transducer 20 inside the transducer holder 100 is fixed while the transducer 20 may vibrate, so that the vibration wave energy loss of the transducer 20 may be minimized. In addition, since the glue is not applied to the front of the transducer 20, the loss of ultrasonic energy radiated forward from the transducer 20 may be prevented. That is, since the flexible glue layer 200 is formed only on the rear surface or the side surface of the transducer 20, it does not cover the front of the transducer 20, and thus there is no restriction on the radiation of ultrasonic energy through the front.

In addition, the space between the transducer holder 100 and the ultrasonic radiation frame 10 is sealed by a sealing member.

The sealing member includes a first sealing member 210 that seals between the head portion 110 of the transducer holder 100 and the front 10a of the ultrasonic radiation frame 10, and a second sealing member 220 that seals between the body portion 120 and the rear 10b of the ultrasonic radiation frame 10.

The first sealing member 210 may include two first and second O-rings 211 and 212 inserted and combined into the rear of the head portion 110. However, the present disclosure is not limited thereto, and the number of the first sealing members 210 may be changed and applied in various ways. In addition, the first sealing member 210 may be applied to any structure that is made of various materials such as silicone and rubber other than an O-ring and may seal.

It is preferable that the first O-ring 211 and the second O-ring 212 are formed with different diameters. The first O-ring 211 and the second O-ring 212 are inserted into a ring-shaped groove 110e formed at the rear of the head portion 110 and are sealed by being pressed against the front surface 10a of the ultrasonic radiation frame 10.

The second sealing member 220 includes a third O-ring 221 that is inserted into the shaft portion 121 of the body portion 120, and an O-ring pressurizing member 222 that is inserted into the shaft portion 121 from the rear of the third O-ring 221 and presses the third O-ring 221 against the rear surface 10b of the ultrasonic radiation frame 10.

The O-ring pressurizing member 222 is formed in a ring shape, and an inclined surface 222a is formed on the front surface so that a part of the third O-ring 221 is settled.

The second sealing member 220 may further include a washer 223 provided between the O-ring pressurizing member 222 and the fastening member 150. The washer 223 is not an essential component of the second sealing member 220 and may be additionally included. The washer 223 may seal the third O-ring 221 and the O-ring pressurizing member 222 and may serve to hold the transducer holder 100.

The second sealing member 220 may be applied to any structure that is made of various materials such as silicone or rubber other than an O-ring or washer and may be sealed.

In addition, a waterproof glue layer 250 is formed between the electrode wire hole 120a of the transducer holder 100 and the electrode wire 180 described below. The waterproof glue may be the same as the flexible glue. In addition, the waterproof glue layer 250 may be formed to fill the entire separation space S with the waterproof glue.

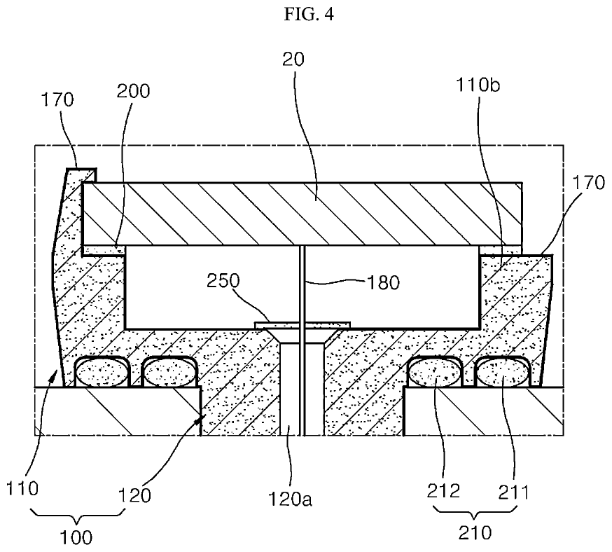

Meanwhile, referring to FIG. 4, the electrode structure using the transducer holder 100 is described as follows.

The surface of the transducer holder 100 is coated with a conductive material to form an electrode 170, and the inside of the transducer holder 100 is formed with a non-conductive material.

The electrode 170 is a coating layer formed by coating the entire surface of the transducer holder 100 with the conductive material, and is grounded. However, the present disclosure is not limited thereto, and the electrode 170 may be formed by coating only a portion of the surface of the transducer holder 100, including a portion that contacts the transducer 20 with the conductive material. In addition, the entire surface of the transducer holder 100 may be coated with the conductive material, and only the surface excluding the inner side of the head portion 110 or the support protrusion 110b may be coated with the conductive material. In the case that the entire surface of the transducer holder 100 is coated with the conductive material, at least a portion of the side surface or the rear surface of the transducer 20 is coated with a non-conductive material to prevent short circuiting. In addition, when at least a portion of the side surface or the rear surface of the transducer 20 is coated with a waterproof material, corrosion and fluctuations in output values due to water intrusion may be prevented.

The conductive material may be any material that may be used as an electrode, such as a metal such as silver. The non-conductive material may be a plastic material.

Therefore, the front and side of the transducer 20 are grounded by contacting the electrode 170, and a current supply unit is connected to the rear of the transducer 20.

The current supply unit may be an electrode wire 180. However, the present disclosure is not limited thereto, and any material that may supply current, such as a pin or connector, may be applied.

The electrode wire 180 is a wire that is connected to the rear center of the transducer 20 by soldering to supply current to the transducer 20.

The electrode wire 180 is arranged to pass through the current supply hole of the transducer holder 100. The current supply hole is an electrode wire hole 120a formed to allow the electrode wire 180 to pass through, as an example.

The electrode wire 180 is extended to the rear of the ultrasonic radiation frame 10 through the electrode wire hole 120a and connected to a separate circuit board.

Here, the electrode 170 is set to one of the positive and negative poles, and the electrode wire 180 is set to the other of the positive and negative poles, so that current may flow to the transducer 20 by the potential difference applied to the electrode 170 and the electrode wire 180. For example, it is also possible that the electrode 170 is set as a positive electrode and the electrode wire 170 is set as a ground electrode, or it is also possible that the electrode 170 is set as a ground electrode and the electrode wire 180 is set as a positive electrode.

The transducer 20 is mounted on the support protrusion 110b, so that the space S is formed between the transducer 20 and the bottom surface of the mounting groove 110a of the transducer holder 100, thereby preventing the electrode wire 180 from contacting the electrode 170, which is the surface of the transducer holder 100, and thus preventing a short circuit.

Therefore, since there is no need to solder the electrode wire to the front surface of the transducer 20, leakage due to the soldering structure on the front surface of the transducer 20 may be prevented. That is, it is possible to prevent leakage from occurring inside the front surface of the transducer 20 that is exposed to the front surface of the ultrasonic radiation frame 10 and contacts the liquid.

In addition, since there is no need to solder the electrode wire to the front surface of the transducer 20, there is an advantage in that the electrode structure is simplified and damage to the transducer 20 may be prevented.

In addition, the high-intensity focused ultrasonic generator configured as described above may prevent leakage from occurring inside from the front surface of the ultrasonic radiation frame 10 by mounting a plurality of transducers 20 on the ultrasonic radiation frame 10 using the transducer holder 100 and sealing the space between the transducers 20 and the transducer holder 100 by bonding them with the flexible glue, even in the case that glue is not applied to the entire front surface of the ultrasonic radiation frame 10.

In addition, since the glue is not applied to the entire front surface of the ultrasonic radiation frame 10, the entire front surface of the transducers 20 is exposed, and thus the loss of ultrasonic energy radiated forward from the transducers 20 may be prevented. In the case where the front surface of the transducers 20 is covered by a glue layer as in the conventional art, there is a problem in that ultrasonic energy is absorbed by the glue layer, but in the present disclosure, since the entire front surface of the transducers 20 is exposed, this may be prevented.

In addition, since the transducer holder 100 is bonded with the flexible glue between the transducer 20 and the transducer 20, the position of the transducer 20 is fixed, and thus the clearance is prevented, while the vibration of the transducer 20 is possible, so that the vibration wave energy loss of the transducer 20 may be reduced.

Furthermore, since the plurality of transducers 20 are individually mounted through the transducer holder 100, and the transducer holder 100 is detachably coupled to the ultrasonic radiation frame 10, there is an advantage in that the transducers 20 may be individually repaired and replaced.

In addition, since the plurality of transducers 20 are individually mounted through the transducer holder 100, there is an advantage in that the capacities of at least some of the plurality of transducers 20 may be configured differently. For example, it is possible to increase the capacities of the transducers arranged on the central side of the ultrasonic radiation frame 10, and of course, it is also possible to control the voltages applied to the plurality of transducers 20 differently.

Furthermore, since the space between the transducer holder 100 and the ultrasonic radiation frame 10 is sealed by a sealing member such as an O-ring, not only may leakage from the front to the rear of the ultrasonic radiation frame 10 be prevented, but there is also an advantage in that the transducer holder may be easily attached or detached from the ultrasonic radiation frame.

Meanwhile, in the above embodiment, it is exemplified that the transducer 20 is attached to all the coupling holes 12 of the ultrasonic radiation frame 10, but the present disclosure is not limited thereto, and it is of course possible to provide the transducer 20 in only at least some of the coupling holes 12 depending on the capacity of the high-intensity focused ultrasound generating device. In the case where the transducers 20 are provided in at least some of the above-described coupling holes 12, the transducer holders 100 are coupled to all the coupling holes 12, and a holder cover for shielding the open front surface may be detachably coupled to some of the transducer holders 100 to which the transducers 20 are not coupled. The holder cover may be formed of a different material from the transducers 20 but in the same shape and may be coupled by glue. Accordingly, the number of the transducers 20 to be mounted may be adjusted, thereby adjusting the energy capacity of the high-intensity focused ultrasound generator.

In addition, in the embodiment, the ultrasonic radiation frame 10 is made of an insulating material such as a resin or a carbon composite, so that the insulation of the ultrasonic radiation frame 10 may be secured. In addition, since there is no need for an additional step of making sure that the ultrasonic radiation frame and the transducer holder do not contact, the number of processes may be reduced and the cost may be reduced.

In addition, when the ultrasonic radiation frame 10 is molded with resin or carbon composite, precision molding is possible, so moldability may be improved, and there is an advantage in that the weight and volume of the ultrasonic radiation frame 10 may be reduced compared to when it is manufactured with other materials such as metal. In the case that the weight of the ultrasonic radiation frame 10 is reduced, the durability of the device for assembling the ultrasonic radiation frame 10 may also be improved.

In addition, by using a material with strong processability and impact resistance, such as polyamide resin, acetal resin, polycarbonate, or polyphenylene oxide, as the insulating material, corrosion resistance, wear resistance, and the like may also be secured.

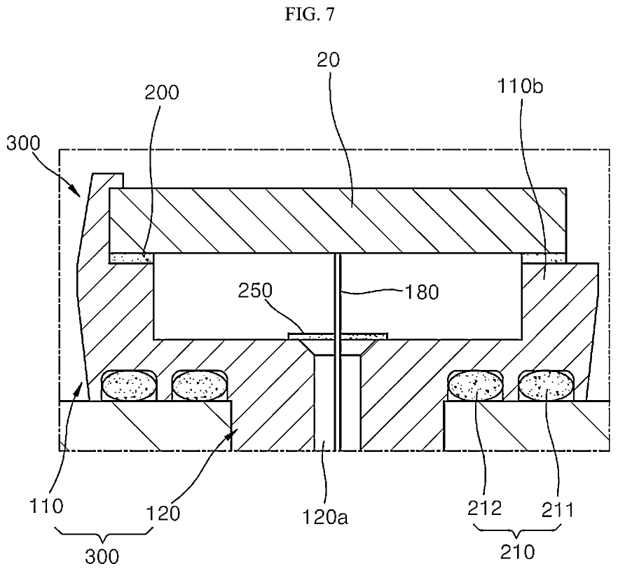

Meanwhile, FIG. 7 is a diagram illustrating an electrode structure using a transducer holder according to a second embodiment of the present disclosure.

Referring to FIG. 7, the electrode structure using the transducer holder according to the second embodiment of the present disclosure is different from the first embodiment in that the entire transducer holder 300 is an electrode formed of a conductive material, and the remaining configuration and operation are the same as the first embodiment, so a detailed description of the similar configuration will be omitted and the differences will be described based on the main points.

The transducer holder 300 is formed of the conductive material and is an electrode itself, and the structure and shape of the first embodiment are applied.

Any material that may be used as an electrode, such as a metal such as silver, may be applied as the conductive material.

The front surface and the side surface of the transducer 20 are grounded by contacting the electrode, and the electrode wire 180 is connected to the rear of the transducer 20.

The electrode wire 180 is a wire that is connected to the center of the rear of the transducer 20 by soldering and supplies current to the transducer 20. The electrode wire 180 is arranged to pass through the electrode wire hole 120a of the transducer holder 100. The electrode wire 180 is extended to the rear of the ultrasonic radiation frame 10 through the electrode wire hole 120a and connected to a separate circuit board.

Here, the transducer holder 300, that is, the electrode, is set to one of the positive and negative poles, and the electrode wire 180 is set to the other of the positive and negative poles, so that current may flow to the transducer 20 by the potential difference applied to the electrode and the electrode wire 180. In addition, it is also possible for the electrode to be set to the ground electrode, and the electrode wire 180 to be set to the positive electrode.

The transducer 20 is mounted on the support protrusion 110b, so that the space S is formed between the transducer 20 and the bottom surface of the mounting groove of the transducer holder 100, thereby preventing the electrode wire 180 from contacting the surface of the transducer holder 300, and thus a short circuit does not occur. In addition, the support protrusion 110b may be coated with a non-conductive material or formed of a non-conductive material.

Therefore, since there is no need to solder the electrode wire to the front surface of the transducer 20, leakage due to the soldering structure at the front surface of the transducer 20 may be prevented. That is, leakage into the interior of the front surface of the transducer 20 that is exposed to the front surface of the ultrasonic radiation frame 10 and contacts the liquid may be prevented.

In addition, since there is no need to solder the electrode wire to the front surface of the transducer 20, there is an advantage in that the electrode structure may be simplified and damage to the transducer 20 may be prevented. In addition, at least a portion of the side surface or the rear surface of the transducer 20 may be coated with a non-conductive material to prevent short circuiting. In addition, at least a portion of the side surface or the rear surface of the transducer 20 is formed by coating with a waterproof material, thereby preventing corrosion or fluctuation of the output value due to water intrusion.

Meanwhile, the high-intensity focused ultrasonic generator according to a third embodiment of the present disclosure is different from the first and second embodiments in that the ultrasonic radiation frame 10 includes a frame body portion formed of metal, and an insulating layer formed by anodizing the surface of the frame body portion. Since the remaining configuration and operation are the same as the first and second embodiments, a detailed description of the similar configuration will be omitted below, and a detailed description will be given focusing on the differences.

Here, the metal is aluminum as an example.

Since the insulating layer is an oxide film formed by anodizing the aluminum, it may improve not only the insulating function of the ultrasonic radiation frame 10, but also the corrosion resistance and wear resistance.

The insulating layer may be formed to a thickness greater than a preset thickness. The preset thickness is set to a thickness that does not conduct current.

Meanwhile, the present disclosure is not limited to the above embodiment, and the frame body portion of the ultrasonic radiation frame 10 may be formed of a material lighter than metal, and the insulating layer may also be a coating layer coated with an insulating material on the surface of the frame body portion.

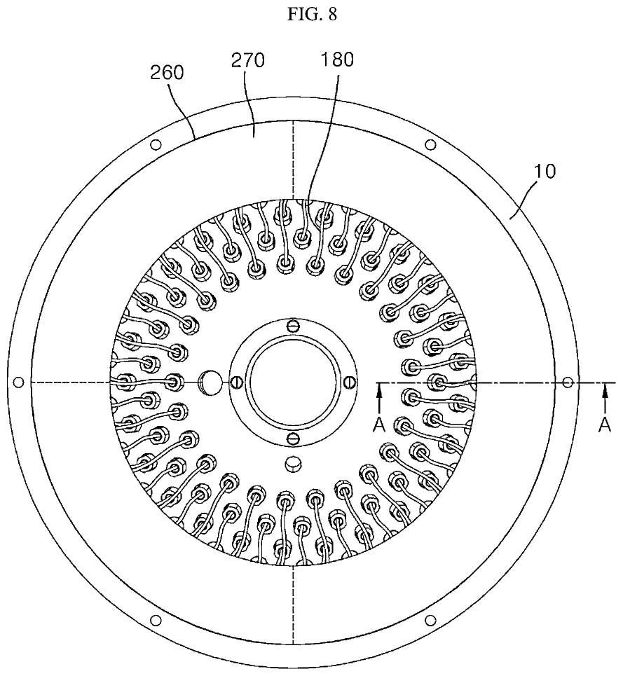

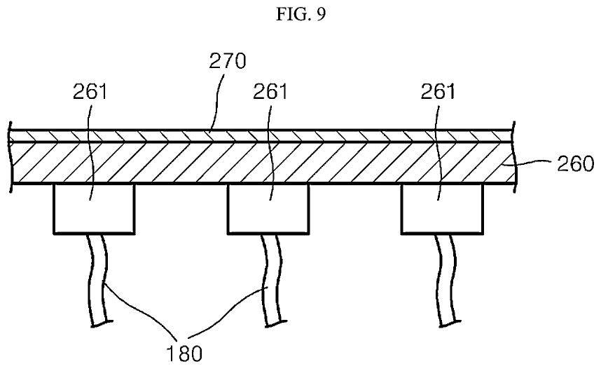

Meanwhile, FIG. 8 is a rear view illustrating an ultrasonic radiation frame and an RF board according to a fourth embodiment of the present disclosure. FIG. 9 is a cross-sectional view taken along line A-A of FIG. 8.

Referring to FIG. 8 and FIG. 9, a high-intensity focused ultrasound generator according to a fourth embodiment of the present disclosure is different from the first, second, and third embodiments in that it further includes an RF board 260 that is provided on the ultrasonic radiation frame 10 and electrically connects the plurality of transducers 20 to each other and supplies RF power to the transducers 20, and the remaining configurations and functions are similar, so the following description will focus on the different configurations and a detailed description of the similar configurations will be omitted.

The transducer holder 100 and the transducer 20 are an integral electrode that is electrically connected by contacting each other. The electrode is connected to the RF board 260 and receive RF power.

The transducer holder 100 is connected to the cathode of the RF board 260 and grounded, and the transducer 20 is connected to the anode of the RF board 260 and receives the RF power, as an example.

The transducer holder 100 and the transducers 20 are each connected to the board connector 261 of the RF board 260 through the electrode wire.

The electrode wire includes a first electrode wire connecting the transducer holder 100 and the board connector 261, and a second electrode wire 180 connecting the transducer 20 and the board connector 261.

The second electrode wire 180 is a wire that is soldered to the center of the rear surface of the transducer 20 to supply RF power to the transducer 20. The second electrode wire 180 is arranged to pass through the electrode wire hole 120a of the transducer holder 100. The second electrode wire 180 is extended to the rear surface of the ultrasonic radiation frame 10 through the electrode wire hole 120a and connected to the RF board 260.

The RF board 260 is detachably connected to the rear of the ultrasonic radiation frame 10, and is connected to a plurality of first electrode wires each connected to the plurality of transducer holders 100, and a plurality of second electrode wires 180 each connected to the plurality of transducers 20.

The RF board 260 is arranged in the remaining part except the center so as to prevent interference with the probe 11 in the case that the probe 11 is coupled to the rear of the ultrasonic radiation frame 10. In addition, the RF board 260 may be formed in multiple pieces. In this embodiment, the RF board 260 is an example in which four of the RF boards 260 are each in an arc shape and are connected to each other to form a ring shape. When the RF boards 260 are composed of multiple pieces, they may be connected to each other, and of course, they may be arranged at a predetermined distance from each other. In addition, the number and shape of the RF boards 260 may be changed in various ways so long as they may prevent interference with the probe 11. In other words, the RF boards 260 may be changed in various ways so long as they are arranged in the remaining part of the ultrasonic radiation frame 10 except for the central portion, which is the joint portion where the probe 11 is coupled. For example, one or more RF boards 260 may be arranged in shapes such as a square, triangle, and crescent shape in the remaining part of the ultrasonic radiation frame 10 except for the central portion.

In addition, when the RF boards 260 are composed of n pieces, the plurality of transducers 20 may be classified into n groups according to their positions, and the plurality of transducers 20 may be connected to n RF boards 260 for each group. Accordingly, the RF board 260 may be individually replaced and repaired.

The RF board 260 is provided with a plurality of board connectors 261.

The board connector 261 is a connector provided on the RF board 260 to which the first and second electrode wires 180 are detachably connected. The board connectors 261 are formed to correspond to the number of the transducers 20 so that the transducers 20 may be independently connected. However, the present disclosure is not limited thereto, and at least two or more transducers 20 may be connected to one board connector 261, and of course, it is also possible for the number of the board connectors 261 to be greater than the number of the transducers 20. In addition, the board connectors 261 may be integrally provided with the RF board 260, or may be detachably coupled to the RF board 260.

The RF board 260 further includes a monitoring sensor.

The monitoring sensor is a sensor provided with the RF board 260 to independently monitor the operating status of the transducers 20. In this embodiment, the monitoring sensor detects the power supply status of the second electrode wires 180 respectively connected to the transducers 20, thereby detecting normal or abnormal operation of the transducers 20, as an example. For example, the monitoring sensor may be a current sensor or a voltage sensor that detects overcurrent or overvoltage or current cutoff of the electrode wires 180. However, the present disclosure is not limited thereto, and any sensor that may detect an abnormal state of the temperature sensor or the transducers 20 may be applied.

An insulating cover 270 is provided on the outer surface of the RF board 260.

The insulating cover 270 is provided to cover the outer surface of the RF board 260 and serves to insulate. In this embodiment, the insulating cover 270 may be a polyimide film as an example, but it is not limited thereto, and any insulating material may be applied. The insulating cover 270 may be connected to the RF board 260 using a fastening member, and the like, or may be attached to the RF board 260 using a separate adhesive member.

Meanwhile, the RF board 260 is connected to a power supply device for supplying the RF power.

The RF board 260 and the power supply device may be connected to the RF board 260 by a plurality of power cables that are detachably connected. The power cables may be Bayonet Neill-Concelman (BNC) cables equipped with BNC connectors, but are not limited thereto and may be implemented in various ways.

As described above, in this embodiment, the transducer 20 is connected to the RF board 260 and receives the RF power through the RF board 260.

Since the plurality of transducers 20 are respectively connected to the board connectors 261 of the RF board 260, in the case that any one of the plurality of transducers 20 is damaged or needs to be replaced, it is possible to repair or replace only the corresponding transducer 20. That is, in the case that the plurality of electrode wires respectively connected to the transducers 20 are tied together and connected to a separate power supply, there is a problem that not only individually checks the status of the transducers, but also individually repairs or replaces them. On the other hand, in the present disclosure, the RF board 260 is provided between the transducers 20 and the power supply, and the second electrode wires 180 connected to the plurality of transducers 20 are individually connected to the RF board 260 through the board connector, so that individual repair or replacement of the transducers 20 may be made available.

In addition, since the state of the plurality of transducers 20 may be individually monitored using the monitoring sensors provided in the RF board 260, only the transducers 20 that need repair or replacement may be identified more easily and quickly, and a response may be made quickly.

Therefore, since the plurality of transducers 20 may be independently monitored and repaired or maintained, maintenance and management may be made easy.

In addition, since the RF board 260 compatible with the probe 11 may be used, it is easy to treat various types of lesions.

Meanwhile, in the embodiment, the transducer holder 100 is grounded and RF power is applied only to the transducer 20 as an example. However, the present disclosure is not limited thereto, and it is also possible to configure the transducer holder 100 to be connected to the cathode of the RF board 260 and the transducer 20 to be connected to the anode of the RF board 260 so that there is a potential difference between the transducer holder 100 and the transducer 20.

Meanwhile, in the embodiments, the case where the surface of the transducer holder 100 is coated with a conductive material or the entire transducer holder 100 is formed with a conductive material so that the transducer holder 100 and the transducer 20 function as an integral electrode was described as an example. However, the present disclosure is not limited thereto, and the transducer holder 100 may of course be formed of a non-conductive material, that is, an insulating material. In the case that the transducer holder 100 is formed of an insulating material, the first and second electrode wires are respectively connected to the upper and lower ends of the transducer 20, and the first and second electrode wires are connected to the RF board 260.

The present disclosure has been described with reference to the embodiments shown in the drawings, but these are merely exemplary, and those skilled in the art will understand that various modifications and equivalent other embodiments are possible from this. Therefore, the true technical protection scope of the present disclosure should be determined by the technical idea of the appended claims.

According to the present disclosure, a high-intensity focused ultrasound generator using an insulating material capable of improving durability and insulation may be manufactured.

The high-intensity focused ultrasonic generator according to the present disclosure has an advantage in that the ultrasonic radiation frame is formed of an insulating material including resin, thereby improving formability and corrosion resistance, and securing electrical insulation, and reducing the weight and volume of the device, so that the cost can be reduced.

In addition, the ultrasonic radiation frame is formed of a carbon composite material that is an insulating material mixed with resin and carbon material, so that the formability, corrosion resistance, and durability, and securing electrical insulation, and reducing the weight and volume of the device, so that the cost can be reduced.

In addition, the ultrasonic radiation frame includes the frame body portion formed of a metal such as aluminum, and an insulating layer formed by anodizing the surface of the frame body portion, so that durability and electrical insulation can be secured.

The present disclosure includes a plurality of transducers individually mounted on the ultrasonic radiation frame by the transducer holder, and at least a portion of the transducer holder is configured with the electrode formed of a conductive material, so that the front of the transducer is electrically connected by contacting the transducer holder, and the rear of the transducer is electrically connected via an electrode wire, so that there is no need to solder the electrode wire to the front of the transducer, thereby preventing leakage due to the soldering structure and making manufacturing easier.

In addition, the transducer is configured to be seated on the support protrusion of the transducer holder, so that the electrode wire coupled to the lower surface of the transducer is prevented from contacting the transducer holder, thereby preventing a short circuit phenomenon, and the electrode structure is stabilized, so that the vibration wave effect can be enhanced.

In addition, since the RF board for applying RF power is provided on the rear of the ultrasonic radiation frame, a plurality of transducers is individually connected to the RF board through board connectors, in the case that some of the plurality of transducers requires repair or replacement, it is easy to repair or replace only the transducer.

In addition, since the monitoring sensor is provided on the RF board, the state of the plurality of transducers can be individually monitored, so there is an advantage in that it is easier to identify transducers that require repair or replacement and respond quickly.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A high-intensity focused ultrasound generator, comprising:
an ultrasonic radiation frame and a coupling hole formed therein;
a transducer holder inserted into the coupling hole of the ultrasonic radiation frame and detachably coupled through the ultrasonic radiation frame; and
a transducer mounted on the transducer holder;
wherein the ultrasonic radiation frame comprises an insulating material;
wherein the transducer holder is an electrode formed of a conductive material or an electrode comprising a surface coated with a conductive material;
wherein at least one of a front surface or a side surface of the transducer is electrically coupled to the transducer holder; and
wherein a rear surface of the transducer has an electrical coupling through a current supply hole formed in a rear surface of the transducer holder.

2. The high-intensity focused ultrasound generator of claim 1, wherein the insulating material comprises:
a resin; or
a carbon composite material including a carbon material mixed with a resin at a preset ratio.

3. The high-intensity focused ultrasound generator of claim 1, wherein the ultrasonic radiation frame includes a frame body portion formed of a metal, and wherein the insulating layer is formed by anodizing a surface of the frame body portion.

4. The high-intensity focused ultrasound generator of claim 1, wherein a surface of the ultrasonic radiation frame includes an insulating layer coated with the insulating material.

5. The high-intensity focused ultrasound generator of claim 1, wherein the transducer holder includes:
a head portion mounted on the ultrasonic radiation frame, wherein the head portion has a mounting groove formed therein into which the transducer is inserted and mounted; and
a body portion that extends rearward from the head portion and penetrates the coupling hole and is coupled by a fastening member at a rear of the ultrasonic radiation frame.

6. The high-intensity focused ultrasound generator of claim 5, wherein the body portion of the transducer holder is formed with the current supply hole, and wherein the current supply hole is configured to allow one or more wires to pass through and be withdrawn to the rear of the ultrasonic radiation frame, and
wherein a space between the one or more wires and the current supply hole is sealed with waterproof glue.

7. The high-intensity focused ultrasound generator of claim 5, wherein the head portion of the transducer holder is formed so that at least a part of a side surface of the mounting groove is opened.

8. The high-intensity focused ultrasound generator of claim 5, wherein the head portion of the transducer holder is formed with a support protrusion that protrudes from a bottom surface of the mounting groove, that supports a lower surface of the transducer, and that forms a space between the transducer and the bottom surface.

9. The high-intensity focused ultrasound generator of claim 8, wherein the support protrusion is formed of a non-conductive material.

10. The high-intensity focused ultrasound generator of claim 5, wherein the head portion of the transducer holder is formed with a catch protrusion that protrudes from a bottom surface of the mounting groove and has a tip bent inward to prevent the transducer inserted into the mounting groove from being detached.

11. The high-intensity focused ultrasound generator of claim 5, wherein the body portion of the transducer holder includes:
a shaft portion that extends rearward from the head portion and is press-fitted into the coupling hole; and
a screw portion that extends rearward from the shaft portion, that penetrates the coupling hole, and that is coupled with the fastening member at the rear of the ultrasonic radiation frame.

12. The high-intensity focused ultrasound generator of claim 1, wherein the side surface and the rear surface of the transducer are coated with at least one of a waterproof material or a non-conductive material.

13. The high-intensity focused ultrasound generator of claim 1, wherein the transducer is one of a plurality of transducers of the high-intensity focused ultrasound generator; and
wherein the high-intensity focused ultrasound generator further comprises an RF board provided on the ultrasonic radiation frame, wherein the RF board is configured to electrically connect the plurality of transducers to each other and to supply RF power to the plurality of transducers.

14. The high-intensity focused ultrasound generator of claim 13,
wherein the RF board further includes one or more board connectors corresponding to one or more wires, and to which the one or more wires are detachably connected.

15. The high-intensity focused ultrasound generator of claim 14, wherein the one or more board connectors are detachably connected to the RF board.

16. The high-intensity focused ultrasound generator of claim 13, further comprising:
a monitoring sensor provided on the RF board and configured to independently monitor operating states of the plurality of transducers by detecting power supply states of wires respectively connected to the transducers.

17. The high-intensity focused ultrasound generator of claim 13, further comprising:
an insulating cover formed to cover an outer side of the RF board.

18. The high-intensity focused ultrasound generator of claim 13, further comprising:
a probe coupled to a center of the ultrasonic radiation frame,
wherein the RF board is provided on a part of the rear of the ultrasonic radiation frame.

19. The high-intensity focused ultrasound generator of claim 13, wherein at least one of the rear surface or the side surface of the transducer and the transducer holder are bonded by an adhesive member so that the transducer is sealed while being vibrated inside the transducer holder; and
wherein the transducer holder and the ultrasonic radiation frame are sealed by a sealing member.

20. A high-intensity focused ultrasound generator, comprising:
an ultrasonic radiation frame having a probe arranged in center and a coupling hole;
a transducer holder inserted into the coupling hole in the ultrasonic radiation frame, wherein the transducer holder is detachably coupled at a rear of the ultrasonic radiation frame; and
a transducer mounted on an open front surface of the transducer holder;
wherein the ultrasonic radiation frame comprises an insulating material,
wherein the transducer holder includes:
a head portion mounted on the ultrasonic radiation frame, wherein the head portion has a mounting groove formed therein into which the transducer is inserted and mounted; and
a body portion that extends rearward from the head portion and penetrates the coupling hole and is coupled by a fastening member at the rear of the ultrasonic radiation frame;
wherein the head portion has a support protrusion that protrudes from a bottom surface of the mounting groove, that supports a lower surface of the transducer, and that forms a space between the transducer and the bottom surface;
wherein the transducer holder is an electrode formed of a conductive material or an electrode comprising a surface coated with a conductive material;
wherein at least one of a front surface or a side surface of the transducer is in contact with the electrode;
wherein a rear surface of the transducer has an electrical coupling through a current supply hole formed in the body portion.
